(19) United States
(12) Patent Application Publication
(10) Pub. No.: US 2019/0216765 A1
(43) Pub. Date: Jul. 18, 2019

(54) COMPOSITIONS AND METHODS OF TREATING FUNGAL INFECTIONS OF THE NAILS

(71) Applicant: KBR Incorporated, San Marcos, CA (US)

(72) Inventor: Kenneth Rehm, San Marcos, CA (US)

(21) Appl. No.: 16/247,953

(22) Filed: Jan. 15, 2019

Related U.S. Application Data
(60) Provisional application No. 62/618,175, filed on Jan. 17, 2018.

(51) Int. Cl. A61K 31/325; A61K 9/70; A61K 47/46; A61K 47/44; A61K 47/26; A61K 47/18; A61K 47/10; A61P 31/10

(52) U.S. Cl. CPC A61K 31/325; A61K 9/70; A61K 47/46; A61P 31/10; A61K 47/26; A61K 47/18; A61K 47/10; A61K 47/44

(57) ABSTRACT

Compositions and methods for reducing the symptoms of skin conditions associated with the nail and surrounding tissue are provided. A medicated pad for cleaning and treating the nail and surrounding tissue is described, wherein said pad is saturated with a therapeutic composition comprising certain botanicals, an antimicrobial agent, a surfactant, and a moisturizing agent. The skin and nail conditions can include fungal infections of the nails. Kits for treating fungal infections of the nails are likewise described. Optionally, the kit may include a hydrating composition which includes shea butter, aloe, vitamins, arnica, emu oil, urea, and Vitamins C, A, E, and D.

### CROSS REFERENCE TO RELATED APPLICATION

This application claims the benefit of U.S. Provisional Application No. 62/618,175, filed on Jan. 17, 2018, the contents of which are hereby incorporated in their entirety.

### BACKGROUND OF THE INVENTION

### Field of the Invention

Embodiments of the invention relate to compositions for treating conditions of the nail and surrounding skin. More particularly, the invention includes compositions and methods for ameliorating the signs and symptoms associated with infections of the nails. Also disclosed are methods of treating the signs and symptoms of fungal infections. Kits comprising an anti-fungal therapeutic are likewise provided.

### Description of the Related Technology

Fungal infection of the nail, also known as onchomycosis or tinea unguium, is a relatively common condition that up to ten percent of all adults in Western countries experience. The percentage of individuals infected with a nail fungus can increase to 20% of adults who are 60 years or older.

Nail fungal infections can be caused by a dermatophyte fungus such as Trichophyton rubrum. Yeasts and molds can also be responsible for fungal infections of the nail. In healthy individuals, a fungal infection of the nails can be transmitted by exposure to moist, wet areas such as communal showers or from nail salons, where instruments are inadequately sanitized. Elderly people as well as people with underlying disease states such as AIDS, diabetes, cancer, psoriasis, or someone taking immunosuppressive medications such as steroids are also at a higher risk of experiencing a fungal infection of the nail.

Typically, fungal infections of the nail can begin as a white or yellow spot on or under the tip of a fingernail or toenail and as the infection progresses, can result in the discoloration, thickening, and deterioration of the nail. Signs of a fungal nail infection can also include a brittle, crumbly or ragged appearance of the nail, a nail which is distorted in shape, as well as a nail bed that is dull. In some cases, the nail can appear dark in color, which is caused by debris building gup under the nail. In other instances, the nail can become loose or lift up as a result of a fungal infection.

While nail infections can pose cosmetic concerns, they are also responsible for causing pain and discomfort due to the changes of the nail. Treatment for fungal nails includes keeping the nail trimmed and filed to reduce the amount of fungus in the nail. Oral antifungal therapy including the use of griseofulvin, tervinafine, itraconazole, or fluconazole has been prescribed to treat fungal nail infections. However, many of the oral therapies only works between about 50-75% of the time and, like many medications, come with unwanted side effects. Even when oral therapy does work, the fungus may come back in about 20-50% of the time. Other treatment protocols include the use of laser therapy or photodynamic therapy, use of electrical current to help absorption of topical antifungal medications, as well as the use of a nail lacquer that alter the microclimate of the nail to make it more difficult for a fungus to grow. Topical medications and creams have likewise been used to treat fungal nail infections but are generally thought to be less effective than oral medications due at least in part to the difficulty in penetrating the hard exterior of the nail. Topical treatments may require daily application for a period of time up to one year in order to see results. These topical treatments include both prescription topical medications such as ciclopirox, efinaconazole, and tavaborole as well as non-prescription treatments such as undecylenic acid and/or propylene glycol, vinegar, and Vicks VapoRub.

Curing fungal nails can be challenging and treatment can take as long as 18 months. Moreover, relapse and reinfection are quite common (40-70% reinfection rate). There remains a need for effective treatments for fungal nail infections which treat the infection without the side effects of an oral medication.

### SUMMARY

Compositions, kits, and methods are described for reducing the symptoms of fungal nail contamination are provided. In one aspect, a medicated pad for use in ameliorating symptoms of conditions of the skin and nail is described. The medicated pad may include a nonwoven disc having a first surface and a second surface; wherein at least one of the first or second surfaces is textured. The medicated pad may further include an aqueous therapeutic composition. The composition can include an effective amount of an antimicrobial agent; an effective amount of a surfactant; a moisturizing agent; and an effective amount of a botanical agent. Advantageously, the nonwoven disc is saturated with the therapeutic composition.

In some aspects, the medicated pad is moistened with antimicrobial agent such as an antifungal agent, an antibacterial agent, or an antiviral agent. The antifungal agent may be tolnaftate. Optionally, the therapeutic composition includes a surfactant such as decyl glucoside or non-ionic polyglucoside.

In another aspect, the medicated pad may include a moisturizer such as allantoin, urea, glycerol triester, or combinations thereof.

The medicated pad advantageously includes a botanical agent such as extract of calendula, chamomile, cornflower, linden leaf, St. Johns wart, garlic, oregano oil, tea tree oil, lavender oil, clove oil, cinnamon oil, and combinations thereof. Optionally, the therapeutic composition may include an effective amount of a disinfectant such as isopropyl alcohol.

In yet another aspect, a system for treating conditions of the skin and nail, are provided. The system may include a container housing a medicated pad having a textured surface; and an aqueous solution of a therapeutic composition. The therapeutic composition may include an effective amount of an antimicrobial agent; an effective amount of a surfactant; a moisturizing agent; and an effective amount of a botanical agent. Advantageously, the system further includes instructions for applying the medicated pad to the surface of a nail bed and surrounding skin to clean the nail bed and surrounding skin surfaces. The botanical agent may include extract of calendula, chamomile, cornflower, linden leaf, St. Johns wart, garlic, oregano oil, clove oil, cinnamon oil, tea tree oil, lavender oil, or combinations thereof.

Optionally, the system can include a second container, which houses a hydrating composition as well as instructions for applying the hydrating agent to the treated surface including the nail bed and surrounding skin after cleaning the nail bed and surrounding skin. The hydrating composition can be massaged into the skin. Advantageously, the hydrating composition includes an effective amount of shea butter; an effective amount of aloe vera powder; an effective amount of arnica; an effective amount of vitamins such as vitamin C, vitamin A, vitamin E, Vitamin B3, Vitamin D and combinations thereof; an effective amount of emu oil; and an effective amount of urea.

In still another aspect, a method of treating a nail infection is provided. The method may include providing a saturated medicated pad having at least one textured surface; wherein the pad is saturated with a therapeutic solution. The therapeutic solution can include an effective amount of an antimicrobial agent; an effective amount of a surfactant; a moisturizing agent; and an effective amount of a botanical agent.

Treatment may further include applying the textured surface of said medicated pad to the surface of a nail and surrounding tissue to clean and debride the surface.

Optionally, the method may include administering a hydrating agent to the surface of the nail and surrounding tissue after cleaning with the medicated pad. The hydrating agent can include an effective amount of shea butter; an effective amount of aloe vera powder; an effective amount of arnica; an effective amount of vitamins such as vitamin C, vitamin A, vitamin E, Vitamin B3, Vitamin D and combinations thereof; an effective amount of emu oil; and an effective amount of urea.

In another aspect, a method of treating a condition of the skin and nails is disclosed. The method may include identifying an individual in need thereof; and applying a medicated pad to the surface of the skin and nails of the individual. The condition may include a bacterial infection, a viral infection, psoriasis, inflammation, pain, and eczematous dermatitis, and combinations thereof.

### BRIEF DESCRIPTION OF THE DRAWINGS

FIG. 1 is a photograph of the left and right foot of an individual presenting with a fungal infection of the toenails.

FIG. 2 is a color photograph of the individual's feet from FIG. 1 after being treated for 28 days with the disclosed composition and kit.

### DETAILED DESCRIPTION

After reading this description, it will become apparent to one skilled in the art how to implement the invention in various alternative embodiments and alternative applications. However, all the various embodiments of the present invention will not be described herein. It is understood that the embodiments presented here are presented by way of an example only, and not limitation. As such, this detailed description of various alternative embodiments should not be construed to limit the scope or breadth of the invention as set forth below.

Embodiments of this application relate to compositions for treating infections of the nail. Also disclosed are medicated pads for treating disorders of the nail. In certain embodiments, compositions, methods and systems for treating fungal infections of the nails are disclosed. Additionally, systems and kits for the treatment of nail infections comprising a plurality of medicated pads and optionally, a moisturizer are provided. Methods of treating fungal infections of the nail with antifungal and/or antibacterial agents are likewise described.

The invention is based, in part, on the surprising discovery of synergistic benefits of a topical therapeutic composition coupled with a nail cleansing pad. This unique combination of therapeutic composition and medicated pad provide both kerolytic properties as well as penetrating properties to reduce the symptoms of nail infections. Thus, in one embodiment, a nail cleansing pad is provided. The nail cleansing pads are designed to clean the surface of the nail by debriding skin and fungal and/or bacterial contaminates while the therapeutic composition conditions and penetrates the surface of a nail including the nail, cuticles, and surrounding skin. The combination of the medicated cleansing pad and therapeutic composition serve to decrease damaging inflammation of the skin and treating the underlying infection while soothing, hydrating, and cleaning the skin and nail plate of microbes such as fungus, bacteria, and viruses. Notably, this cleaning is accomplished without harming the protective acid mantle layer of the skin. The medicated pads serve to generate a healthy pH and aid in the removal of hard dead skin while increasing the skin's permeability to the therapeutic composition.

The invention can include a system for treating nail infections comprising a container and a plurality of cleansing and debriding pads which are saturated with an active therapeutic composition hereinafter described. As used herein, saturated is meant to include wetted, impregnated, moistened, or any other similar term to convey that the pad is exposed to the therapeutic composition in such a way as to absorb at least a portion of the therapeutic composition on the pad surface such that a previously dry pad is made moist to become a medicated pad. The plurality of pads are configured to be stacked inside the container with a therapeutic composition which impregnates into the pads by capillary action. The pad is advantageously highly absorbent, non-linting, disposable, and debriding. It may be constructed of either woven or nonwoven material. In a preferred embodiment, the pad is constructed of a nonwoven material such as a non-woven cotton. The pad may be inserted in the container through the open end and a therapeutic composition is introduced into the container so that it is absorbed by the pad. The open sided container may further include a sealing layer (not shown) to seal the saturated pads with the therapeutic composition and to prevent drying.

The nail cleansing pads of the present invention are configured to be impregnated with the therapeutic composition and absorb at least a portion of the therapeutic composition. Additionally, the pads provide surface for scrubbing the nail and surrounding tissue of an individual. Preferably, the pads comprise a nonwoven material fused together to form a multiple layer medicated pad. Pads of the present invention can have a plurality of textural differences such as smooth nonwoven material fused to a coarse nonwoven material to create varying degrees of textural differences. In one embodiment, the cleansing pad is disc shaped. However, it will be appreciated by a person of skill in the art that the pad can be in square, rectangular, or any other suitable shape.

Although not limited thereto, in some aspects, the present invention includes nonwoven fabrics derived from “oriented” or carded fibrous webs composed of textile-length fibers, the major proportion of which are oriented predominantly in one direction. Exemplary of such fibers are the natural fibers such as cotton and wool and the synthetic or man-made cellulosic fibers, notably rayon or regenerated cellulose, such as those supplied by BASF.

Other fibers of a synthetic or man-made origin may be used such as: polyamide fibers such as nylon 6, nylon 66, nylon 610, etc.; polyester fibers such as “Dacron”, “Fortrel” and “Kodel”; acrylic fibers such as “Acrilan”, “Orlon” and “Creslan”; modacrylic fibers derived from polyethylene and polypropylene; cellulose ester fibers such as “Arnel” and “Acele”; polyvinyl alcohol fibers, etc.

Methods of making nonwoven cloths are not a part of this invention and, being well known in the art, are not described in detail herein. Generally, however, such cloths are made by air- or water-laying processes in which the fibers or filaments are first cut to desired lengths from long strands, passed into a water or air stream, and then deposited onto a screen through which the fiber-laden air or water is passed. The deposited fibers or filaments are then adhesively bonded together, using the resins of the present invention, dried, cured, and otherwise treated as desired to form the nonwoven cloth.

The preferred nonwoven cloth substrates used in the invention herein are generally adhesively bonded fibers or filamentous products having a web or carded fiber structure (when the fiber strength is suitable to allow carding) or comprising fibrous mats in which the fibers or filaments are distributed haphazardly or in random array (i.e., an array of fibers in a carded web where partial orientation of the fibers is frequently present, as well as a completely haphazard distributional orientation), or substantially aligned. The fibers or filaments can be natural (e.g., wool, silk, jute, hemp, cotton, linen, sisal, or ramie) or synthetic (e.g., rayon, cellulose ester, polyvinyl derivatives, polyolethins, polyamides, or polyesters) as have been described hereinabove.

The absorbent properties preferred herein are particularly easy to obtain with nonwoven cloths and are provided merely by building up the thickness of the cloth, i.e., by superimposing a plurality of carded webs or mats to a thickness adequate to obtain the necessary absorbent properties, or by allowing a sufficient thickness of the fibers to deposit on the screen. Any denier of the fiber (generally up to about 15 denier) can be used, inasmuch as it is the free space between each fiber that makes the thickness of the cloth directly related to the absorbent capacity of the cloth. Thus, any thickness necessary to obtain the required absorbent capacity can be used.

The cleansing pads are combined with a therapeutic composition, which is in solution, such that the cleansing pads are moistened, impregnated, or otherwise saturated with the therapeutic composition as will be described in greater detail below. The therapeutic composition is advantageously in aqueous solution and is readily absorbed by the pads such that the pads are moistened. In some embodiments, the therapeutic composition is formulated as an oil and is likewise readily absorbed by the pads such that the pads are moistened.

The cleansing pads can be used as a cleansing cloth to wipe away dirt, oils, and odor-causing bacteria and fungus from the surface of the skin in an individual in need thereof. Additionally, when used to clean the nail surface and surrounding surfaces, the pad aids in making the skin and cuticles softer as well as helping to loosen debris for more effective nail management. Moreover, the textured surface of the pad is adapted to soothe and gently clean contaminants that thrive in between dry, cracked, scaly skin and in between the toes. In other embodiments, the pad is adapted to clean the surface of the nail and to soften the nails, which facilitates easy and safer nail trimming.

The active therapeutic composition for which the cleansing pads are saturated is advantageously formulated in an aqueous solution. In another embodiment, the composition is formulated in an oil solution. The composition is formulated to clean and moisturize the nail surface as well as the surrounding tissue, treat microbes such as fungus, bacteria and viruses, and lower the pH of the treated surface from a pH of 7 to a pH of around 6.5, 6.0, 5.5, or 5. The reduction in pH promotes the delivery of antimicrobial agents, moisturizers, fragrance, botanicals, or combinations thereof into the skin. In some aspects, the composition comprises an effective amount of citric acid to reduce the pH to around 5.5.

In one embodiment, the therapeutic composition comprises about 75% purified water. In some embodiments, the composition comprises greater than 76%, 77%, 78%, 79%, 80%, 81%, 82%, 83%, 84%, 85%, 86%, 87%, 88%, 89%, or 90% purified water. In one embodiment, the therapeutic composition comprises about 85% purified water. The therapeutic composition can further include a surfactant, preferably a mild non-ionic alky polyglucoside surfactant such as capryl glucoside, coco glucoside, lauryl glucoside, decyl glycoside or combinations thereof in an amount of between about 5 to 20% of the formulation. In one embodiment, the surfactant is decyl glucoside in an amount of around 10% w/w of the formulation.

The therapeutic composition also includes an antimicrobial agent to remove fungus, bacteria and/or viruses from the surface of a nail. The antimicrobial agent can include a topical antibiotic selected from the group consisting of sulfacetamide sodium, bacitracin/polymyxin B, erythromycin, silver sulfadiazine, neomycin, retapmulin, mupirocin, mafenide, tolnaftate, clotrimazole, econazole nitrate, miconazole, terbinafine, fluconazole, ketoconazole, amphotericin, benzoic acid, undecyclenic acid, nystatin, naftifine, ciclopirox, efinaconazole, terbinafine, tavaborole, penciclovir, acyclovir, and combinations thereof. The antimicrobial agent is present in an amount of between about 0.1 and 5% w/w of the composition. In one embodiment, the antimicrobial agent is tolnaftate in an amount of about 1.0% w/w of the composition. The antimicrobial agent can be a botanical agent such as oregano oil, clove oil, cinnamon oil, tea tree oil or combinations thereof, which possess antibacterial, antiviral, and antifungal properties. The clove oil may be in the amount of about 10% w/w of the formulation. In some embodiments, the therapeutic composition comprises about 1% w/w of oregano oil to treat foot or nail fungus. In another embodiment, the therapeutic composition comprises an effective amount of an antimicrobial agent such as tolnaftate and a natural antimicrobial agent selected from the group consisting of oregano oil, clove oil, tea tree oil, cinnamon oil, or combinations thereof.

The therapeutic composition further includes a moisturizing agent. In one embodiment, the moisturizing agent is allantoin, urea, or combinations thereof. Allantoin helps skin to lock in moisture and encourages skin to shed dead cells as well as grow new ones, which promotes healthy cell development and prevents dull, sallow skin. It can soften the horny substance (keratin) that holds the top layer of skin cells together, which helps the dead skin cells fall off, helps the skin to keep in more water, and leaves the skin feeling smoother and softer. Allantoin conditions, moisturizes, protects, soothes, and promotes healing. In one embodiment, allantoin is present in the composition in an amount of between about 0.05 to about 5 w/w of the composition. In another embodiment, the composition comprises about 0.1% w/w allantoin. Urea is a humectant which helps the skin maintain a healthy moisture balance, keeping it soft, supple, youthful, and bringing relief to a dry skin. Urea reduces roughness on a skin surface by maintaining skin hydration. When urea is applied to the skin, it penetrates the stratum corneum, where it readily absorbs and retains water, thus increasing the capacity of the skin to hold moisture and rehydrate. In addition to improving skin moisture, urea is associated as having anti-itch properties and provide a natural exfoliant. Urea has been shown to increase the skin permeability of certain skin care ingredients, working as a vehicle for other performance ingredients, encouraging them to penetrate the epidermis easily. Urea can be present in the composition in an amount of between about 0.05-1.0% w/w. In some embodiments, the composition comprises about 0.1% w/w urea. In another embodiment, the moisturizing agent is an effective amount of glycerol triester.

In some embodiments, the therapeutic composition includes an effective amount of a preservative. Advantageously, the preservative is paraben-free and formaldehyde-free. The preservative can include phenoxyethanol, caprylyl glycol, and combinations thereof. In one embodiment, the preservative is optiphen. The preservative is present in an amount between about 0.1 to 1.0% w/w of the composition. In another aspect, the composition comprises about 0.75% w/w optiphen.

The composition can further include an effective amount of a disinfectant. The disinfectant can be an alcohol such as isopropyl alcohol or a botanical agent having disinfecting properties. Suitable botanical agents include, without limitation, lemongrass, citrus, lavender, bergamot, tea tree oil, essential oils, grapefruit extract, rosemary, cinnamon, eucalyptus, lemon, and/or clove. The botanical agents can include between about 1-10% w/w of lavender, eucalyptus, oregano, lemongrass, or cinnamon oil or combinations thereof. In a preferred embodiment, the formulation comprises about 5.0% w/w of the formulation. In still another embodiment, the disinfectant can be white distilled vinegar. The disinfectant can be present in an amount of between 0.1 to 2.0% w/w of the composition. In one embodiment, the disinfectant is isopropyl alcohol and is present in the amount of about 0.5% w/w of the composition.

The therapeutic composition for use in treating nail infections can further include an effective amount of an essential oil such as tea tree oil (Malaleuca alternifolia), lavender oil (Lavandula angustifolia), or combinations thereof. An effective amount of tea tree oil and lavender oil is generally between about 0.1 to about 1% w/w, more preferably between about 0.25% to about 0.5% w/w of the composition. Tea tree oil may be applied to the skin for its natural anti-inflammatory and antiseptic properties. Moreover, tea tree oil is associated with the treatment of infections such as fungal infections of the nail, lice, scabies, athlete's foot, and ringworm. It is believed to be effective at least in part because it helps to remove dry and dead skin cells including psoriatic plaques in individuals suffering from psoriasis. It also possesses antibacterial, antimicrobial, antiviral, antifungal, antiseptic, and anti-inflammatory properties which work to relieve itching, redness, and burning. Moreover, it is believed to control the overproduction of skin cells. Lavender oil also benefits the skin due to its antimicrobial and antioxidant characteristics, bringing rapid healing to dry skin as well as cuts and scrapes.

In yet another aspect, the therapeutic composition can comprise an effective amount of one of a plurality of botanical agents which include, without limitation: extracts of calendula, chamomile, cornflower, linden leaf, St. John's wart, garlic, and coneflower. Briefly, extract of calendula possesses antiviral activity as well as serves as an anti-inflammatory for the skin. Linoleic acid which is found in high concentrations of calendula is a powerful anti-inflammatory agent. Calendula-based gels and topical ointments help to speed recovery and healing, improve skin firmness, promote hydration, increase blood flow and oxygen to infected areas, and act as an anti-fungal, astringent, and healing promoter. Chamomile possesses many powerful skin properties such as anti-inflammatory, anti-fungal, antibacterial, antiseptic, and contains essential oils and antioxidants. Additionally, it is hypoallergenic and helps to reduce skin irritants by neutralizing free radicals. Similarly, cornflower has anti-inflammatory properties and promotes wound healing. It is also antibacterial and is a good anti-oxidant, astringent, and soothes inflamed and irritated wounds and skin. Linden leaf is both a demulcent and astringent, making it an effective remedy for excessive dryness. The demulcent qualities add moisture to the body, while the astringent qualities tighten and tone tissues, helping to keep the moisture in the skin. St. Johns wort has antibacterial, antioxidant, and antiviral properties. Because of its anti-inflammatory properties, it has been applied to the skin to help heal wounds and burns. St. Johns Wort is very effective in relieving sore muscles and therefore sore feet, muscle strain, and tension. Garlic possesses antiviral characteristics as well as being an effective antifungal agent. Garlic contains diallyl sulphide (DAS) and diallyl disulphide (DADSO, which significantly inhibit proteinase, phospholipase secretion, and dimorphism in Candida albicans. These compounds can, therefore, act as a potent anti-fungal in the management of candidiasis. The botanical agents can be present in the therapeutic composition in an amount of between about 0.05 to 5% w/w of the composition. In certain embodiments, the botanical agent or agents are present in an amount of between about 0.025-0.30% w/w.

In one embodiment, the therapeutic composition can include an effective amount of cannabidiol (CBD) oil. CBD oil, the non-intoxicating marijuana extract, possesses anti-fungal, antibacterial, antiviral, and anesthetic properties. Advantageously, CBD oil is present in an amount between about 1-20% w/w of the formulation. In a preferred embodiment, the amount of CBD oil is between about 5-10% w/w of the composition. In a particularly preferred embodiment, the CBD oil is about 10% w/w of the formulation. In another aspect, CBD oil is replaced with an effective amount of ginger root oil, clove bud oil, or combinations thereof.

In some embodiments, the system or kit can further include a separate container comprising a hydrating composition as described below. Thus, it is contemplated that the system/kit can include both a container comprising the medicated pads as well as a separate container comprising a hydrating composition for applying to the affected area after the application of the medicated pad to a nail surface. The kit includes instructions for first cleaning the infected nail surface with a medicated pad as described above and then moisturizing the surrounding tissue with a hydrating composition to prevent the area from becoming dry, improving permeability, and promoting penetration of the epidermis by the active ingredients of the medicated pad. Optionally, the instructions can further include directions to massage the hydrating composition into the skin to reduce stiffness and fatigue, promote circulation, and hasten the absorption of therapeutic composition and/or hydrating composition.

The medicated cleansing pads prepares the affected surface, removes microbes and dead skin, treats the underlying infection, and lowers the pH of the skin surface to enhance the permeability of the skin and nails to a separate hydrating composition such as the Balm Riche All Natural Foot Cream™ moisturizer or Balm Riche All Natural Diabetic Foot Cream™ moisturizer (Dr. Rehm Remedies, San Marcos, Calif.). Advantageously, a suitable hydrating composition can includes, without limitation, water, aloe vera, xanthan gum, allantoin, sodium borate, lactic acid, vitamin C/ascorbic acid, tocopherol, a preservative such as phenoxyethenol, caprylyl glycol, potassium sorbate and/or hexylene glycol, Vitamin B3, Vitamin A, Vitamin D, shea butter, coconut oil, high oleic safflower oil, emu oil, glyceryl stearate, cetaryl alcohol, stearic acid, beeswax, granulated lecithin, tea tree oil, lavender oil, and botanicals such as chamomile, fever few, arnica, marigold, lime tree, St. Johns Wort and combinations thereof. Exemplary percentages of the effective amounts of the various hydrating components can be found in Table 1.

TABLE 1
Balm Riche Formula

| | | Percentage |
|---|---|---|
| Ingredient/Common Name | INCI | (% w/w) |
| Water | Water | 35-55 |
| Aloe Vera Powder | Aloe barbadensis leaf | 0.05-1.0 |
| Xantham Gum | Xantham Gum | 0.05-1.5 |
| Allantoin | Allantoin | 0.05-5.0 |
| Borax | Sodium Borate | .25-2.0 |
| Lactic Acid | Lactic Acid | 0.2-2.0 |
| Citric Acid | Ascorbic Acid | .05-1.0 |
| Vitamin E Mixed Tocopherols | Tocopherol | 0.05-0.5 |
| Cap 5 Preservatives | Phenoxyethanol, caprylyl | 0.05-2.0 |
| | glycol, potassium sorbate, | |
| | hexylene glycol | |
| Vitamin B3 | Niacinimide | 0.01-1.0 |
| Vitamin A | Retinyl palmitate | 0.05-0.5 |
| Vitamin D | Cholecalciferol | 0.05-0.5 |
| Shea Butter | Butyrospermum parkii | 1-5 |
| Coconut Oil | Cocos nucifera | 1-10 |
| High oleic safflower oil | Carthamus tinctorius | 1-15 |
| Emu oil | Emu oil | 1-5 |
| Glyceryl stearate | Glyceryl stearate | 1-5 |
| Cetearyl alcohol | Ceteryl alcohol | 0.5-5 |
| Stearic acid | Stearic acid | 0.5-5 |
| Beeswax | Cera alba | 0.25-10.0 |
| Lecithin granules | Lecithin | 0.05-1.0 |
| Tea Tree Oil | Malaleuca alternifolia | 0.25-25.0 |
| Lavendar Oil | Lavandula angustifolia | 0.25-5.0 |
| Chamomile | Matricaria recutita flower | 0.10-5.0 |
| Fever few | Chrysanthemum parthenium | 0.10-5.0 |
| | oil | |
| Arnica | Arnica montana | 0.10-5.0 |
| Marigold | Helianthus anuus, calendula | 0.10-5.0 |
| | officinalis | |
| Lime tree | Citrus aurantifolio oil | 0.10-5.0 |
| CBD oil | Canabis sativa | 5.0-10% |
| St. Johns Wort | Hypericum perforatum | 0.10-5.0 |

A method of treating a nail infection is also provided. The method includes providing a medicated pad such as a pad treated with a therapeutic composition as described herein and cleaning the nailbed and surrounding tissue. One or more pads can be used to remove hard dead skin from the surface of the affected nail and surrounding tissue as well as to clean the surface of the nail bed while providing an antimicrobial treatment. As described above, the medicated pad is soaked, saturated, or otherwise treated with a therapeutic composition comprising an effective nail infection treatment amount of: an antifungal agent such as tolnaftate, botanicals selected from the group consisting of: chamomile, calendula, coneflower, linden leaf, oregano, garlic, St. Johns Wort, tea tree oil, lavender oil and combinations thereof; a disinfectant such as isopropyl alcohol; allantoin, urea, citric acid, optiphen, and combinations thereof. The disinfectant can include a fatty or wax alcohol such as lauryl, cetearyl, stearyl, and cetyl alcohol. In other embodiments, the disinfectant may be one of various glycols or isopropanol. Other disinfectants may include, without limitation, an effective amount of ethanol, ethyl alcohol, denatured alcohol, methanol, isopropyl alcohol, SD alcohol, benzyl alcohol, or combinations thereof. The pad removes debris and dead skin and the therapeutic composition treats the underlying infection, lowering the pH of the treatment surface, reducing inflammation, and increasing permeability of anti-infective agents such as an antifungal agent to expedite resolution of the infection. In use, the medicated pad serves to debride the surface of the nail and surrounding skin to remove fungal contaminates, the therapeutic composition penetrates the surface of the treated area to hasten absorption of the therapeutic agent and reduce microbial populations; and the cream serves to soften the treated area. The softening of the nail and surrounding tissue facilitates debridement as well as providing a more pliant nail. Some of the hallmarks of fungal nail infections is the yellowing and extreme hardening of the nail. Successful resolution of nail infections often requires that the infected/contaminated nail be trimmed to remove the yellowed areas attendant to infection. However, with hardened nails, it can be quite difficult to accomplish this trimming. By applying a cream such as the one detailed above as an adjuvant to the medicated pads with a therapeutic composition, the user is able to treat symptoms of the underlying infection, clean the nail surface, and soften the skin and nails to enable more effective trimming and debridement.

Optionally, the method of treatment further includes applying a hydrating composition to the affected nail surface and surrounding tissue after treatment with a medicated pad. The moisturizing agent prevents dryness around the nail surface as well as the surrounding tissue and promotes healing. The moisturizer can include, without limitation, one or more of the following components: glycerin, ceramides, urea, glycolic acid, hyaluronic acid, lactic acid, occlusive such as dimethicone, petrolatum, paraffin, and lanolin or combinations thereof, humectants, emollients, as well as natural botanicals such as aloe vera, chamomile, fever few, arnica, marigold lime tree, and St. John's Wort and combinations thereof, and essential oils such as tea tree oil, lavender oil, and combinations thereof. In one embodiment, the moisturizing agent is Balm Riche All Natural Foot Cream™ moisturizer or Balm Riche All Natural Diabetic Foot Cream™ moisturizer (Dr. Rehm Remedies, San Marcos, Calif.).

A system for reducing the symptoms skin conditions associated with the feet and hands is likewise contemplated. More particularly, skin conditions contemplated for treatment with the above-described medicated pad include any condition for which debridement and moisturizing of the affected area ameliorate symptoms of the condition. Exemplary conditions contemplated include, without limitation psoriasis, fungal infections such as ringworm and athlete's foot, eczema, and eczematous dermatitis. The condition can further include a bacterial infection, a viral infection, inflammation, pain, and combinations thereof.

### Example 1

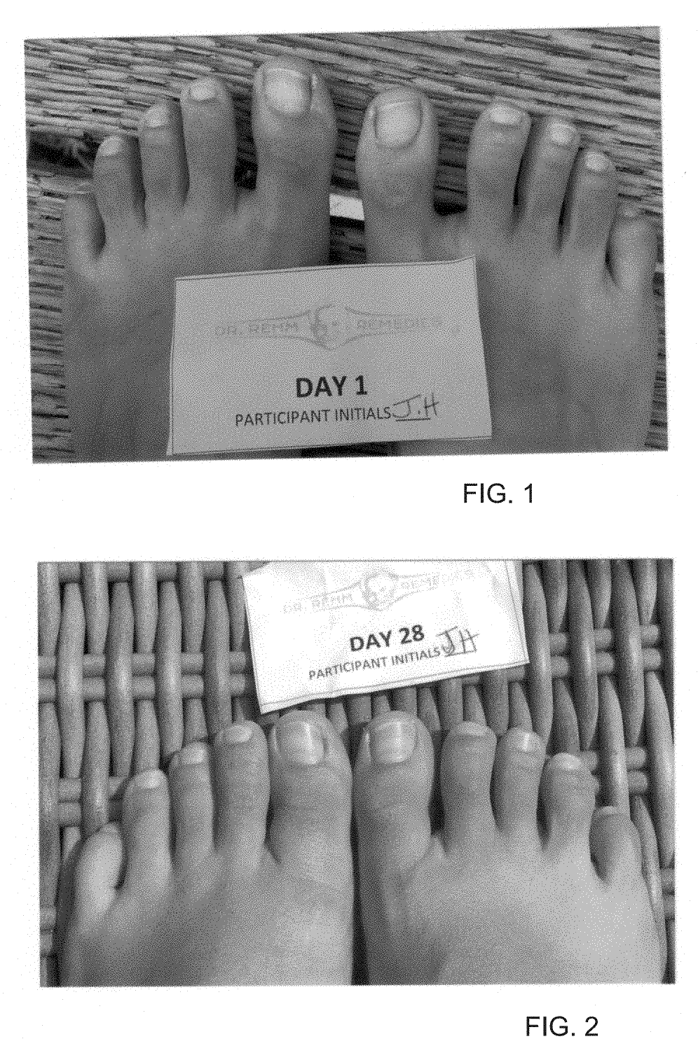

The efficacy of the disclosed kit for ameliorating signs of fungal contaminants and infections of the toe nails was studied. An individual presenting with dry skin and infected nail beds was identified. A photograph of the individual's untreated feet is set forth in FIG. 1. Notably, the individual's nails are discolored. The cuticles on the untreated toes are thick and in need of debridement. The surface of at least the left big toe had noticeable ridges and the surrounding tissue was dry and crusty.

The individual treated the surface of the nails and surrounding tissue with medicated pads moistened with a therapeutic solution as described above. The textured medicated pad was used to scrub the surface of the nails and surrounding tissue and reduce the amount of dead skin and fungal contaminants. After 28 days, a second photograph was taken as illustrated in FIG. 2. Notice that the signs and symptoms of fungal infection on the toenails have been dramatically reduced. The nail beds are clean and no longer discolored. The surrounding skin is likewise clean and moisturized. The cuticles and nails have been softened.

The above description of disclosed embodiments is provided to enable any person skilled in the art to make or use the invention. Various modifications to the embodiments will be readily apparent to those skilled in the art; the generic principles defined herein can be applied to other embodiments without departing from spirit or scope of the invention. Thus, the invention is not intended to be limited to the embodiments shown herein but is to be accorded the widest scope consistent with the principles and novel features disclosed herein.

What is claimed is:

1. A medicated pad for use in ameliorating symptoms of conditions of the skin and nail, comprising:
a nonwoven disc having a first surface and a second surface; wherein at least one of said first or second surfaces is textured; and
an aqueous therapeutic composition; said composition comprising:
an effective amount of an antimicrobial agent;
an effective amount of a surfactant;
a moisturizing agent; and
an effective amount of a botanical agent;
wherein said nonwoven disc is saturated with said therapeutic composition.

2. The medicated pad of claim 1, wherein said antimicrobial agent is selected from the group consisting of: an antifungal agent, an antibacterial agent, and an antiviral agent.

3. The medicated pad of claim 2, wherein said antifungal agent is tolnaftate.

4. The medicated pad of claim 1, wherein said surfactant is a non-ionic alky polyglucoside.

5. The medicated pad of claim 4, wherein said surfactant is decyl glucoside.

6. The medicated pad of claim 1, wherein said moisturizer is selected from the group consisting of allantoin, urea, and combinations thereof.

7. The medicated pad of claim 1, wherein said botanical agent is selected from the group consisting of extract of calendula, chamomile, cornflower, linden leaf, St. Johns wart, garlic, oregano oil, tea tree oil, lavender oil, clove oil, cinnamon oil, CBD oil, and combinations thereof.

8. The medicated pad of claim 1, further comprising an effective amount of a disinfectant.

9. The medicated pad of claim 8, wherein said disinfectant is isopropyl alcohol.

10. A system for treating conditions of the skin and nail, comprising:
a container, comprising:
a medicated pad having a textured surface; and
an aqueous solution of a therapeutic composition, said composition comprising:
an effective amount of an antimicrobial agent;
an effective amount of a surfactant;
a moisturizing agent; and
an effective amount of a botanical agent; and
instructions for applying said medicated pad to the surface of a nail bed and surrounding skin to clean the nail bed and surrounding skin surfaces.

11. The system of claim 10, further comprising a second container, said second container comprising a hydrating composition; and said instructions further comprising directions to apply said hydrating agent to said nail bed and surrounding skin after cleaning said nail bed and surrounding skin.

12. The system of claim 11, wherein said instructions further comprise instructions to massage said hydrating agent into the nail bed and surrounding skin surfaces.

13. The system of claim 10, wherein said antimicrobial agent is selected from the group consisting of: an antifungal agent, an antibacterial agent, and an antiviral agent.

14. The system of claim 10, wherein said antifungal agent is tolnaftate.

15. The system of claim 10, wherein said surfactant is a non-ionic alky polyglucoside.

16. The system of claim 15, wherein said surfactant is decyl glucoside.

17. The system of claim 10, wherein said moisturizer is selected from the group consisting of allantoin, urea, glycerol triester, and combinations thereof.

18. The system of claim 10, wherein said botanical agent is selected from the group consisting of extract of calendula, chamomile, cornflower, linden leaf, St. Johns wart, garlic, oregano oil, tea tree oil, lavender oil, CBD oil, and combinations thereof.

19. The system of claim 11, wherein said hydrating composition comprises:
an effective amount of shea butter;
an effective amount of aloe vera powder;
an effective amount of arnica;
an effective amount of vitamins selected from the group consisting of vitamin C, vitamin A, vitamin E, Vitamin B3, Vitamin D and combinations thereof;
an effective amount of emu oil; and
an effective amount of urea.

20. A method of treating a nail infection, comprising:
providing a saturated medicated pad; said medicated pad having at least one textured surface; wherein said pad is saturated with a therapeutic solution comprising:
an effective amount of an antimicrobial agent;
an effective amount of a surfactant;
a moisturizing agent; and
an effective amount of a botanical agent;
applying the textured surface of said medicated pad to the surface of a nail and surrounding tissue to clean said surfaces.
